(19) United States
(12) Patent Application Publication
(10) Pub. No.: US 2012/0214705 A1
(43) Pub. Date: Aug. 23, 2012

(54) BETA-DEFENSIN 2 GENETIC VARIATION PREDICTS H. PYLORI SUSCEPTIBILITY

(75) Inventors: Charles L. Bevins, Davis, CA (US); Jay V. Solnick, Davis, CA (US); Michael J. Hornsby, South San Francisco, CA (US); Robert J. Kays, Burlingame, CA (US)

(73) Assignee: The Regents Of The University Of California Office of Technology,, Oakland, CA (US)

(21) Appl. No.: 13/497,765

(22) PCT Filed: Sep. 27, 2010
(86) PCT No.: PCT/US10/50435

§ 371 Date: May 6, 2012

Related U.S. Application Data
(60) Provisional application No. 61/246,449, filed on Sep. 28, 2009.

(51) Int. Cl. C40B 30/04; C12Q 1/68

(52) U.S. Cl. 506/9; 435/6.12

(57) ABSTRACT

The present invention relates to correlating high gene copy number and/or the overexpression of β-defensin 2 (“BD2”) with susceptibility to disease conditions resulting from, associated with or mediated by Helicobacter pylori infection, for example, susceptibility to peptic ulcer, non-ulcer dyspepsia, gastric cancer (e.g., gastric adenocarcinoma), and mucosa associated lymphoid tissue (MALT) lymphoma.

### CROSS-REFERENCE TO RELATED APPLICATIONS

This application is a U.S. national phase filing under 35 U.S.C. §371 of PCT/US2010/050435, filed on Sep. 27, 2010, which claims the benefit of U.S. Provisional Application No. 61/246,449, filed on Sep. 28, 2009, all of which are hereby incorporated herein by reference for all purposes.

### STATEMENT AS TO RIGHTS TO INVENTIONS MADE UNDER FEDERALLY SPONSORED RESEARCH AND DEVELOPMENT

This invention was made with Government support under Grant Nos. AI032738, AI050843, AI042081 and AI060555, awarded by the National Institute of Health. The Government has certain rights in this invention.

### FIELD OF THE INVENTION

The present invention relates to correlating high gene copy number and/or the overexpression of β-defensin 2 (“BD2”) with susceptibility to disease conditions resulting from Helicobacter pylori infection, for example, susceptibility to peptic ulcer or gastric cancer.

### BACKGROUND OF THE INVENTION

H. pylori is an important human pathogen that infects the stomach of approximately one-half the world's population [Suerbaum, S and Michetti, P, (2002). Helicobacter pylori infection. N Engl J Med 347: 1175-1186]. The clinical outcome of infection is variable. Although most of those infected will have no clinical sequelae, 5 to 10% will develop peptic ulcer, and about 1 to 3% (˜50 million individuals worldwide) will progress to gastric cancer, the second most frequent cause of cancer-related death. For many reasons, including cost factors, treating all asymptomatic carriers of H. pylori with antibiotics is neither feasible nor recommended. However, since only some individuals with H. pylori infection will develop serious clinical sequelae, much effort is focused on identifying predictors of clinical disease. To date, most of these investigations have addressed bacterial factors, such as the cag pathogenicity island (cag PAI), which is a well-studied bacterial locus linked to H. pylori-associated disease. Little is currently known about what host factors might predict clinical outcome of H. pylori infection. Herein, a common genetic variation in defensins useful in predicting susceptibility to H. pylori disease is described. The variation is detectable by a relatively simple assay of genomic DNA obtained from blood or saliva samples.

Defensins are key effector molecules of the innate immune system, serving as endogenous antibiotics that protect all mucosal surfaces of the body. These peptides are found in virtually every animal species tested, from humans to horseshoe crabs; related molecules are found in other invertebrates and even plants. This widespread distribution, together with data from studies in model systems, provide compelling evidence that defensins are central to host defense against infectious disease.

In humans and in the non-human primate Rhesus macaque model, induction of beta-defensin 2 (official gene name is DEFB4 (also known as HBD4), but referred to herein as BD2, to be consistent with peptide name, beta-defensin-2) is fundamental to the mucosal innate immune response to initial infection with H. pylori. Studies of the rhesus macaque that BD2 induction is a hallmark of the cag PAI-dependent mucosal response to H. pylori infection have been recently reported [Hornsby, et al., (2008) Gastroenterol 134: 1049-1057]. Studies of human gastric biopsies and cultured epithelial cell models also indicate that infection with cag PAI-positive H. pylori results in a robust induction of BD2 [Boughan, et al., (2006) J Biol Chem 281: 11637-48; O'Neil, et al., (2000) Infect Immun 68: 5412-5415; and Wada, A, (1999) Biochem Biophys Res Commun 263: 770-774.].

In the studies herein of Rhesus macaques as an animal model of H. pylori infection, the fact that these non-human primates have extensive variation in gene copy number (GCN) of the gene encoding BD2 has been discovered, which is highly similar to that seen in humans. The data that follow are consistent with the conclusion that variation in BD2 GCN is reflected in expression levels of BD2, and that these differences affect the outcome of infection with H. pylori. Although the scope of the current data is limited to the effects of BD2 GCN on bacterial load and gastric inflammation during acute H. pylori infection, these are surrogate markers that are thought to affect the different clinical outcomes that occur after long-term infection with H. pylori in humans. Therefore, the studies herein demonstrate that in human populations, variability in defensin GCN predicts genetic susceptibility to different clinical outcomes of H. pylori infection.

### BRIEF SUMMARY OF THE INVENTION

The present invention provides methods of determining the risk of an individual for developing a pathological disease conditions resulting from an Helicobacter pylori infection. Accordingly in one aspect, the invention provides methods of determining an increased susceptibility or increased risk of an individual to a disease condition mediated by, associated with or secondary to Helicobacter pylori infection. In some embodiments, the methods comprise determining in a biological sample from the individual the gene copy number (GCN) of the β-defensin 2 (BD2) gene, wherein a high GCN of the BD2 gene is indicative of an increased susceptibility of the individual to the disease condition mediated by, associated with or secondary to the H. pylori infection.

In some embodiments, a GCN of the BD2 gene that is 5, 6, 7, 8, 9, 10, or higher, is indicative of an increased susceptibility or increased risk of the individual to develop a disease condition mediated by, associated with or secondary to the H. pylori infection.

In some embodiments, the disease condition mediated by, associated with or secondary to the H. pylori infection is selected from the group consisting of peptic ulcer, non-ulcer dyspepsia, gastric cancer (e.g., gastric adenocarcinoma), and mucosa associated lymphoid tissue (MALT) lymphoma.

In some embodiments, the methods further comprise the step of obtaining a biological sample. In some embodiments, the biological sample is blood or saliva. In some embodiments, the biological sample is gastric tissue or intestinal tissue. In some embodiments, the biological sample is a hair bulb. In some embodiments, the biological sample is a solid tissue sample.

In some embodiments, the individual has or has had an H. pylori infection. In some embodiments, the individual is suspected of having an H. pylori infection. In some embodiments, the individual has a family history of one or more disease conditions mediated by, associated with or secondary to the H. pylori infection, e.g., peptic ulcer, non-ulcer dyspepsia, gastric cancer (e.g., gastric adenocarcinoma), and mucosa associated lymphoid tissue (MALT) lymphoma.

In some embodiments, the GCN is determined by PCR, for example, by real-time quantitative PCR or paralog ratio tests. In some embodiments, the GCN is determined by fluorescent in situ hybridization (FISH). In some embodiments, the GCN is determined by a method selected from the group consisting of PCR, fluorescent in situ hybridization, multiplex amplifiable probe hybridization (MAPH), multiplex ligation-dependent probe amplification (MLPA), dynamic allele-specific hybridization (DASH), array hybridization and mass spectroscopy, or any other techniques known to those skilled in the art.

In some embodiments, the methods further comprise the step of determining the expression of BD2 in gastric tissue or intestinal tissue in the individual, wherein the increased expression of BD2 in gastric tissue or intestinal tissue is indicative of an increased susceptibility or increased of the individual to the disease condition mediated by, associated with or secondary to the H. pylori infection. In some embodiments, the expression level of the BD2 gene is determined. In some embodiments, the expression level of the BD2 protein is determined. In some embodiments, the expression level can be compared to, e.g., the expression levels of BD2 mRNA or protein expression in an individual not infected with H. pylori or an individual with a low BD2 GCN, a BD2 GCN of 4 or fewer. In some embodiments, the expression level of BD2 mRNA or protein expression is compared to a threshold level, e.g., from a population of individuals not infected with H. pylori or an individual with a low BD2 GCN, a BD2 GCN of 4 or fewer.

In some embodiments, the methods further comprise the step of determining bacterial load of H. pylori in the individual, wherein a high bacterial load is indicative of an increased susceptibility of the individual to the disease condition mediated by, associated with or secondary to the H. pylori infection. In some embodiments, the H. pylori bacterial load can be compared to, e.g., the H. pylori bacterial load in an individual with a low BD2 GCN, e.g., a BD2 GCN of 4 or fewer.

In a related aspect, the invention provides methods for determining an increased susceptibility of an individual to a disease condition mediated by, associated with or secondary to an Helicobacter pylori infection. In some embodiments, the methods comprise determining in a biological sample from the individual the expression level of BD2, wherein a high expression level of BD2 is indicative of an increased susceptibility of the individual to the disease condition mediated by, associated with or secondary to the H. pylori infection. In some embodiments, the expression level of the BD2 mRNA is determined. In some embodiments, the expression level of the BD2 protein is determined. In some embodiments, the expression level can be compared to, e.g., the expression levels of BD2 mRNA or protein expression in an individual not infected with H. pylori or an individual with a low BD2 GCN, a BD2 GCN of 4 or fewer. In some embodiments, the expression level of BD2 mRNA or protein expression is compared to a threshold level, e.g., from a population of individuals not infected with H. pylori or an individual with a low BD2 GCN, e.g., a BD2 GCN of 4 or fewer.

In some embodiments, the methods further comprise the step of obtaining a biological sample. In some embodiments, the biological sample is gastric tissue or intestinal tissue.

In some embodiments, the individual has or has had an H. pylori infection. In some embodiments, the individual is suspected of having an H. pylori infection. In some embodiments, the individual has a family history of one or more disease conditions mediated by, associated with or secondary to the H. pylori infection, e.g., peptic ulcer, non-ulcer dyspepsia, gastric cancer (e.g., gastric adenocarcinoma), and mucosa associated lymphoid tissue (MALT) lymphoma.

In some embodiments, the methods further comprise determining in a biological sample from the individual the gene copy number (GCN) of the β-defensin 2 (BD2) gene, wherein a high GCN of the BD2 gene is indicative of an increased susceptibility of the individual to the disease condition mediated by, associated with or secondary to the H. pylori infection.

In some embodiments, a GCN of the BD2 gene that is 5, 6, 7, 8, 9, 10, or higher, is indicative of an increased susceptibility of the individual to the disease condition mediated by, associated with or secondary to the H. pylori infection.

In some embodiments, the disease condition mediated by, associated with or secondary to the H. pylori infection is selected from the group consisting of peptic ulcer, non-ulcer dyspepsia, gastric cancer (e.g., gastric adenocarcinoma), and mucosa associated lymphoid tissue (MALT) lymphoma.

In some embodiments, the GCN is determined by PCR, for example, by real-time quantitative PCR or paralog ratio tests. In some embodiments, the GCN is determined by fluorescent in situ hybridization (FISH). In some embodiments, the GCN is determined by a method selected from the group consisting of PCR, fluorescent in situ hybridization, multiplex amplifiable probe hybridization (MAPH), multiplex ligation-dependent probe amplification (MLPA), dynamic allele-specific hybridization (DASH), array hybridization and mass spectroscopy, or any other techniques known to those skilled in the art.

In some embodiments, the methods further comprise the step of determining bacterial load of H. pylori in the individual, wherein a high bacterial load is indicative of an increased susceptibility of the individual to the disease condition mediated by, associated with or secondary to the H. pylori infection.

In some embodiments the determinations of GCN of the BD2 gene, BD2 mRNA and/or protein expression levels and/or H. pylori bacterial load are recorded in a tangible medium, for example, on paper, in a computer readable format (on a CD, DVD, memory stick, etc.).

In some embodiments, the individual, patient or subject is a mammal, for example, a human, a non-human primate, a laboratory mammal (mouse, rat, rabbit, hamster), a domesticated mammal (feline, canine), or an agricultural mammal (equine, bovine, ovine, porcine).

The invention further provides kits comprising reagents for determining the GCN of the BD2 gene, BD2 mRNA and/or protein expression levels and/or H. pylori bacterial load and instructions for correlating a high BD2 GCN and/or high BD2 mRNA and/or protein expression levels with an increased susceptibility for a disease mediated by, associated with or secondary to an H. pylori infection.

In a related aspect, the invention provides methods for identifying a candidate for treatment of a disease condition mediated by, associated with or secondary to an Helicobacter pylori infection, comprising: a) providing a biological sample of from a candidate subject; b) detecting a gene copy number (GCN) for the BD2 gene in said biological sample; and c) identifying said candidate subject as suitable for treatment with an inhibitor of BD2.

In some embodiments, the subject has an H. pylori infection or has had an H. pylori infection.

In some embodiments, the methods further comprise the step of obtaining a biological sample. In some embodiments, the biological sample is blood or saliva. In some embodiments, the biological sample is a hair bulb. In some embodiments, the biological sample is a solid tissue sample.

In some embodiments, a GCN of the BD2 gene that is 5 or higher identifies said candidate subject as suitable for treatment with an inhibitor of BD2.

In some embodiments, the disease condition mediated by, associated with or secondary to the H. pylori infection is selected from the group consisting of peptic ulcer, non-ulcer dyspepsia, gastric cancer (e.g., gastric adenocarcinoma), and mucosa associated lymphoid tissue (MALT) lymphoma.

In some embodiments, the GCN is determined by PCR, for example, by real-time quantitative PCR or paralog ratio tests. In some embodiments, the GCN is determined by fluorescent in situ hybridization (FISH). In some embodiments, the GCN is determined by a method selected from the group consisting of PCR, fluorescent in situ hybridization, multiplex amplifiable probe hybridization (MAPH), multiplex ligation-dependent probe amplification (MLPA), dynamic allele-specific hybridization (DASH), array hybridization and mass spectroscopy, or any other techniques known to those skilled in the art.

Further embodiments are as described herein.

### DEFINITIONS

Biological samples refer to the solid tissue or a biological fluid that contains either a BD2 nucleic acid or expressed BD2 protein, with or without the GCN polymorphism. With respect to nucleic acids, the biological sample can be tested by the methods described herein and include body fluids including whole blood, serum, plasma, cerebrospinal fluid, urine, lymph fluids, and various external secretions of the respiratory, intestinal and genitourinary tracts, tears, saliva, milk, white blood cells, and the like; and biological fluids such as cell extracts, cell culture supernatants; fixed tissue specimens; and fixed cell specimens. Biological samples can also be from solid tissue, including hair bulb, skin, gastric tissue, intestinal tissue, biopsy or autopsy samples or frozen sections taken for histologic purposes. These samples are well known in the art. A biological sample is obtained from any individual to be tested for the BD2 GCN polymorphism. A biological sample can be suspended or dissolved in liquid materials such as buffers, extractants, solvents and the like.

Structurally, a BD2 or wild-type BD2 (also known as SAP1; DEFB2; HBD-2; DEFB-2; DEFB102; DEFB4; DEFB4A) refers to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, or more amino acids, or over the full-length, to an amino acid sequence encoded by a BD2 nucleic acid (see, e.g., GenBank Accession No: NM_{—}004942.2); (2) bind to antibodies, e.g., polyclonal antibodies, raised against an immunogen comprising an amino acid sequence of a BD2 polypeptide (e.g., encoded by a nucleic acid sequence of GenBank Accession No: NM_{—}004942.2 or having the amino acid sequence of, e.g., GenBank Accession No. NP_{—}004933.1), and conservatively modified variants thereof; (3) specifically hybridize under stringent hybridization conditions to an anti-sense strand corresponding to a nucleic acid sequence encoding a BD2 protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, or over the full-length, to a BD2 nucleic acid (e.g., GenBank Accession No: NM_{—}004942.2).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine I, Lysine (K);

5) Isoleucine (I), Leucine (L), Methionine (M), Valine (V);

6) Phenylalanine (F), Tyrosine (Y), Tryptophan (W); and

7) Serine (S), Threonine (T)

(see, e.g., Creighton, Proteins (1984)).

An antibody refers to either a polyclonal or monoclonal antibody that is able to recognize a specified protein. Antibodies may be generated that are able to recognize either an entire protein, or short peptide sequences within a full length protein.

The terms “bind(s) specifically” or “specifically bind(s)” or “attached” or “attaching” refers to the preferential association of an anti-BD2 antibody, in whole or part, with a cell or tissue bearing a particular target epitope (i.e., a BD2 polypeptide) in comparison to cells or tissues lacking that target epitope. It is, of course, recognized that a certain degree of non-specific interaction may occur between an antibody and a non-target epitope. Nevertheless, specific binding, may be distinguished as mediated through specific recognition of the target epitope. Typically specific binding results in a much stronger association between the delivered molecule and an entity (e.g., an assay well or a cell) bearing the target epitope than between the bound antibody and an entity (e.g., an assay well or a cell) lacking the target epitope. Specific binding typically results in greater than about 10-fold and most preferably greater than 100-fold increase in amount of bound anti-BD2 antibody (per unit time) to a cell or tissue bearing the target epitope as compared to a cell or tissue lacking the target epitope. Specific binding between two entities generally means an affinity of at least 10⁶ M⁻¹. Affinities greater than 10⁸ M⁻¹ are preferred. Specific binding can be determined using any assay for antibody binding known in the art, including Western Blot, ELISA, flow cytometry, immunohistochemistry.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., a BD2 polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50-100 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to BD2 nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)).

Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “threshold level” refers to a representative or predetermined expression level of BD2 polypeptide or mRNA. The threshold level can represent expression detected in a sample from a normal control, i.e., from non-cancerous tissue, usually of the same tissue type of the biological sample subject to testing. The threshold level can be determined from an individual or from a population of individuals. In the present diagnostic methods, BD2 expression, at the transcriptional or translational level, above the threshold level is generally indicative of the presence of susceptibility to H. pylori infection and diseases mediated by, associated with or secondary to H. pylori infection; binding below the threshold level is generally indicative of reduced or lack of susceptibility to H. pylori infection and diseases mediated by, associated with or secondary to H. pylori infection.

The term “increased expression level” is generally made with reference to a predetermined threshold level or a level of expression from a normal or non-cancerous control. An increased expression level is determined when the level of expression in the test biological sample is at least about 10%, 25%, 50%, 75%, 100% (i.e., 1-fold), 2-fold, 3-fold, 4-fold or greater, in comparison to the predetermined threshold level of expression or the level of expression from a normal or non-cancerous control tissue. In determining an increased level of expression, usually the same tissue types are compared.

The term “patient,” “subject,” “individual,” interchangeably refer to a mammal, for example, a human or non-human primate, a domesticated mammal (e.g., feline or canine), an agricultural mammal (e.g., ovine, bovine, equine, porcine) or a laboratory mammal (e.g., mouse, rat, rabbit, hamster).

### BRIEF DESCRIPTION OF THE DRAWINGS

FIG. 1—Distribution of BD2 GCN variation in a control population. The β-defensin gene cluster on human chromosome 8 has dramatic GCN polymorphisms in specific β-defensin genes, including BD2. Within the population, some individuals harbor two copies per genome of BD2, whereas others have up to twelve gene copies [Hollox, et al., (2003). Am J Hum Genet 73: 591-600; and Linzmeier, R M and Ganz, T, (2005). Genomics 86: 423-430.]. The average is 4 copies of BD2 gene per genome in most individuals.

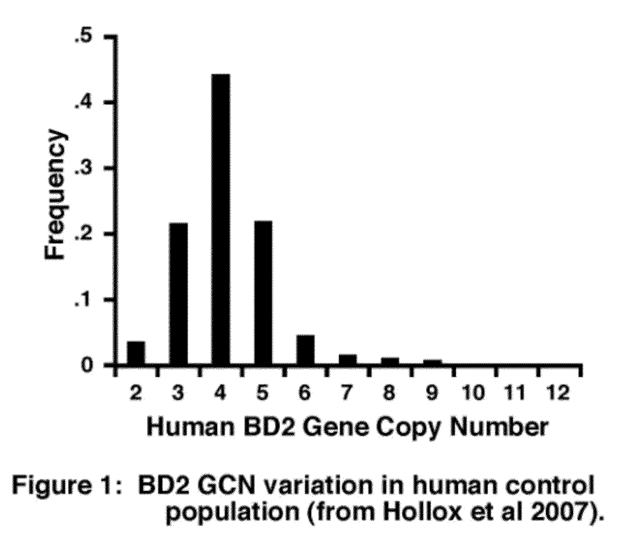

FIG. 2—Microarray analysis of mRNA shows BD2 is the major gene expressed in H. pylori-infected tissue of rhesus macaques compared to uninfected controls for all differentially expressed genes with p<0.01. Each gene (n=119) is represented along the vertical axis and its absolute fold change at 13 wks post inoculation is shown along the horizontal axis. The BD2 mRNA peak is indicated. SPF rhesus macaques were inoculated with WT H. pylori and with an isogenic knockout (KO) strain in which the cag PAI was deleted [Hornsby, et al., (2008). Gastroenterol 134: 1049-1057]. Quantitative cultures of three antral biopsies at 1, 4, 8, and 13 wks post inoculation showed that all challenged animals (but not controls) were infected, typically with 10⁵ to 10⁷ CFU/g of gastric tissue. Sections of gastric histopathology demonstrated that monkeys infected with WT H. pylori had gastritis typical of that seen in humans, while the inflammation was much reduced in the cag PAI KO infected animals. Microarray analysis was performed on gastric mucosa at 13 weeks post inoculation and on uninfected control animals. A key observation was the induction of BD2 mRNA, which was induced ˜11-fold.

FIG. 3—Quantitative RT-PCR analysis of BD2 mRNA expression in H. pylori-infected stomach tissue and controls. RNA was isolated from gastric biopsies obtained from rhesus monkeys before and 1, 4, 8, and 13 weeks after infection with H. pylori WT (black) or cag PAI KO (white). β-actin control was not different between groups.

FIG. 4—GCN determination for BD2 and beta globin in 32 rhesus macaques. Real-time quantitative PCR assays utilized rhesus-specific primers from highly conserved regions of BD2, with beta globin as a diploid control gene (90% nucleotide identity with human). Blue arrow indicates value for diploid gene. The role of BD2 GCN in the host response to H. pylori infection was examined by detecting BD2 GCN variation in 32 randomly selected rhesus macaques. Genomic DNA was extracted from blood samples, and used quantitative real-time PCR to determine BD2 GCN. As a control and reference, values for β-globin as a diploid gene were determined. In contrast to the GCN of ˜2 per diploid consistently observed for β-globin, the BD2 GCN varied from ˜2 to ˜13 among these animals. This range of BD2 GCN is similar to that observed in humans (2-12 copies/diploid).

FIG. 5—(A) Scattergram of BD2 GCN and H. pylori bacterial load; (B) Scattergram of BD2 mRNA transcript abundance and H. pylori bacterial load. Immunohistochemistry of gastric tissue stained with antibody to rhesus BD2 in a monkey with 8 (C) or 2 (D) copies of the BD2 gene per diploid. The H. pylori challenge of rhesus macaques (N=5) was repeated and examined to determine the relationship between BD2 GCN and H. pylori bacterial load in the gastric antrum and corpus. H. pylori colony forming units (CFU) was positively correlated (R=0.47) with BD2 GCN (FIG. 5A). A positive correlation (R=0.70) between H. pylori CFU and BD2 mRNA transcript abundance was also found (FIG. 5B), which was confirmed by immunohistochemistry with anti-BD2 antibody staining of gastric tissue from an animal with 8 BD2 gene copies (FIG. 5C) compared to one with 2 BD2 gene copies (FIG. 5D). These data are clearly consistent with the conclusion that BD2 increases the fitness of H. pylori, for example, by selectively suppressing competing gastric biota.

FIG. 6—Antimicrobial activity of recombinant rhesus BD2 against H. pylori J166 and several other bacteria isolated from the rhesus stomach. H. pylori was less sensitive to BD2 than the other bacteria.

### DETAILED DESCRIPTION

1. Introduction

Gene copy number (GCN) variation is a newly described phenomenon, which likely contributes significant phenotypic variability within populations. Substantial GCN variation is present in genes that encode defensins, cationic antimicrobial peptides that play an important role in innate immunity to infectious diseases. The gene encoding beta-defensin 2 (variably named to as DEFB2; HBD-2; DEFB-2; DEFB102; DEFB4 or DEFB 4A, but referred to herein as BD2), which is a key part of the innate immune response in skin and mucosal surfaces, varies from 2-12 copies per diploid genome. Recent evidence has linked variation in the BD2 GCN with susceptibility to idiopathic diseases: low GCN increases the risk for Crohn's disease of the colon, while high GCN is associated with increased risk of psoriasis. The rhesus macaque model was used to demonstrate that, like in humans, BD2 expression is induced by Helicobacter pylori, a common gastric pathogen that is the causative agent of peptic ulcer and increases the risk for gastric cancer (e.g., gastric adenocarcinoma). Furthermore, preliminary data suggest that rhesus macaques, like humans, have marked variation in BD2 GCN. Variation in BD2 GCN is reflected in expression levels of BD2, and these differences affect the pathogenic or non-pathogenic outcome of infection with H. pylori. Since only about 5 to 10% of humans infected with H. pylori will have clinical sequelae, while the remainder will have only asymptomatic gastritis, there is considerable interest in understanding host factors that are associated with disease. Understanding the functional relationship between genetic variations in the BD2 gene and H. pylori infection will therefore not only expand the knowledge of the relationship between the innate immune response and infection, but also provides a translational link to better understand who can benefit from treatment of H. pylori in order to prevent peptic ulcer and gastric cancer (e.g., gastric adenocarcinoma).

2. Subjects Who Can Benefit from the Present Methods

Any mammal can benefit from the present methods of determining BD2 gene copy number and/or BD2 expression levels and correlating with susceptibility to H. pylori infection and diseases mediated by, associated with or secondary to H. pylori infection. Usually, the mammal is a human.

In various embodiments, the subject may presently have or may have had an H. pylori infection or a disease mediated by, associated with or secondary to an H. pylori infection, e.g., peptic ulcer, non-ulcer dyspepsia, gastric cancer (e.g., gastric adenocarcinoma), and/or mucosa associated lymphoid tissue (MALT) lymphoma. Alternatively, the subject may have a family member (e.g., a parent, a sibling, a grandparent) who has a BD2 gene copy number of 5 or more, or who presently has or has had an H. pylori infection or a disease mediated by, associated with or secondary to an H. pylori infection. The subject can be symptomatic or asymptomatic of an H. pylori infection or a disease mediated by, associated with or secondary to an H. pylori infection.

In some embodiments, the subject is exhibiting symptoms of or is suspected of having an H. pylori infection or a disease mediated by, associated with or secondary to an H. pylori infection, e.g., peptic ulcer, non-ulcer dyspepsia, gastric cancer (e.g., gastric adenocarcinoma), and/or mucosa associated lymphoid tissue (MALT) lymphoma.

The present diagnostic methods find use in conjunction with presently available diagnostic tests for H. pylori infection or diseases mediated by, associated with or secondary to H. pylori infection. The patient may already have a preliminary diagnosis of H. pylori infection or a disease mediated by, associated with or secondary to H. pylori infection, e.g., based on a serum biomarker or a genetic analysis.

3. Methods of Determining Increased Susceptibility to an H. pylori Infection

a. Obtaining a Biological Sample

The present methods may include the step of first obtaining a biological sample from the subject to be tested for susceptibility to diseases mediated by, associated with or secondary to H. pylori infection. The biological sample can be a fluid sample or a solid tissue sample, and contains a nucleic acid (genomic or mRNA) encoding BD2 protein or BD2 protein. For the purposes of determining BD2 gene copy number, the biological sample comprises genomic DNA. For the purposes of determining the expression levels of the BD2 gene at the level of transcription, the biological sample comprises mRNA. For the purposes of determining the expression levels of BD2 at the level of protein, the biological sample comprises BD2 protein.

In some embodiments, the biological sample is a solid tissue sample, for example, a hair bulb, tissue from a cheek swab, or a biopsy. For the purposes of determining BD2 gene copy number, the solid tissue sample can be from any tissue. When correlating BD2 expression levels with disease susceptibility and/or severity mediated by, associated with or secondary to H. pylori infection, the tissue sample can be from the tissue affected by the H. pylori infection. In various embodiments, the solid tissue sample is gastric tissue or intestinal tissue. In some embodiments, a feces sample can be tested. Alternatively, the biological tissue sample can be a fluid tissue sample, e.g., from blood, tears, saliva, serum, or urine. Fluid samples may find use in instances where the BD2 protein levels correlate with the BD2 gene copy number.

b. Determining the Gene Copy Number of β-Defensin 2 (BD2) Gene

The number of copies of the BD2 gene present in an individual can be determined using any method known in the art. Techniques for determining gene copy number are well known in the art and find use in the present methods. Methods for the determination of gene copy number are discussed, e.g., in Barnes, Methods Mol Biol. (2010) 628:1-20; Gru, et al., Breast Cancer Res Treat. (2010), PMID: 20814818; Fanciulli, et al., Clin Genet. (2010) 77(3):201-13; Vissers, et al., J Med Genet. (2010) 47(5):289-97; Henrichsen, et al., Hum Mol Genet. (2009) 18(R1):R1-8; Zhang, et al., Annu Rev Genomics Hum Genet. (2009) 10:451-81; Shen, et al., BMC Genet. (2008) 9:27; Cooper, et al., Nat Genet. (2008) 40(10):1199-203; McCarroll, et al., Nat Genet. (2008) 40(10):1166-74; and Komura, et al., Genome Res. (2006) 16(12):1575-84.

Illustrative methods for detecting gene copy number include without limitation, PCR, e.g., quantitative PCR, high-resolution microarray platforms, sequencing, fluorescent in situ hybridization (FISH), multiplex amplifiable probe hybridization (MAPH), multiplex ligation-dependent probe amplification (MLPA), dynamic allele-specific hybridization (DASH), array hybridization and mass spectroscopy, or any other techniques known to those skilled in the art.

In one embodiment, the gene copy number is detected in genomic DNA samples by quantitative PCR. The following exemplary human primers can be used:

(SEQ ID NO: 1)
BD2 Forward: AGGCGATACTGACACAGGGTTTGT
(SEQ ID NO: 2)
BD2 Reverse: GGAGACCACAGGTGCCAATTTGTT
(SEQ ID NO: 3)
BD2 Forward: ATCAGCCATGAGGGTCTTGT
(SEQ ID NO: 4)
BD2 Reverse: GGAGGGGAATGAGAGGAGAC

Illustrative methods for determining gene copy number of human defensins is described in Linzmeier and Ganz, Genomics (2005) 86(4):423-30, and in Linzmeier and Ganz, Genomics (2006) 88(1):122-6. Real-time PCR can be performed (e.g., using a Roche LightCycler or similar instrument). This technique continually monitors the cycle-by-cycle accumulation of fluorescence signal (monitored at 530 nm) from intercalation of the SYBR green probe into the PCR product (e.g., analyzed using LightCycler Software (Roche Diagnostics)). For each experiment, data points are run in triplicate for the target (BD2) and reference genes as described (Linzmeier, 2005, supra). Accordingly, quantification is performed by the comparative C_{T} (threshold cycle) method. Copy number (CN) of the target BD2 gene will be calculated by first subtracting the threshold cycle number (C_{T}) for the BD2 gene from that of the reference gene. From the difference in threshold cycle, ΔC_{T}=C_{T (reference)}−C_{T (target)}, gene copy number per diploid genome will be calculated using the formula: CN (target)=2 (ΔCt)×CN (reference). Estimated gene copy number is the average of the results obtained for each sample and using each of two primer sets for the target gene and two reference genes.

As a reference diploid gene, the copy number of myeloperoxidase gene can be determined using the following exemplary primers:

(SEQ ID NO: 5)
MPO Forward: CCAGCCCAGAATATCCTTGG
(SEQ ID NO: 6)
MPO Reverse: TGGTGATGCCTGTGTTGTCG
(SEQ ID NO: 7)
TBP Forward: TGAGAAGATGGATGTTGAGTTG
(SEQ ID NO: 8)
TBP Reverse: AGATAGCAGCACGGTATGAG

Single copy (diploid) reference genes TATA box binding protein (“TBP”) and myeloperoxidase (“MPO”) were chosen because they have no known pseudogenes. Both reference genes are amplified simultaneously, but in separate wells for every DNA sample. Two separate copy number assays can be carried out for each individual sample.

A gene copy number of the BD2 gene that is 4 or fewer, e.g., 1, 2, 3, or 4, indicates that the individual is relatively less susceptible to, less likely to, or not at risk of developing a disease condition mediated by, associated with or secondary to the H. pylori infection, e.g., peptic ulcer, non-ulcer dyspepsia, gastric cancer (e.g., gastric adenocarcinoma), and mucosa associated lymphoid tissue (MALT) lymphoma, e.g., in comparison to an individual with a BD2 gene copy number that is greater than 4. Generally, the lower the gene copy number of BD2, the lower the susceptibility or risk of developing a disease condition mediated by, associated with or secondary to the H. pylori infection, e.g., peptic ulcer, non-ulcer dyspepsia, gastric cancer (e.g., gastric adenocarcinoma), and mucosa associated lymphoid tissue (MALT) lymphoma.

Conversely, a gene copy number of the BD2 gene that is 5 or greater, e.g., 5, 6, 7, 8, 9, 10, 11, 12, 13, or higher, indicates that the individual is relatively more susceptible to, more likely to, or at risk of developing a disease condition mediated by, associated with or secondary to the H. pylori infection, e.g., peptic ulcer, non-ulcer dyspepsia, gastric cancer (e.g., gastric adenocarcinoma), and mucosa associated lymphoid tissue (MALT) lymphoma, e.g., in comparison to an individual with a BD2 gene copy number that is fewer than 5. Generally, the higher the gene copy number of BD2, the higher the susceptibility or risk of developing a disease condition mediated by, associated with or secondary to the H. pylori infection, e.g., peptic ulcer, non-ulcer dyspepsia, gastric cancer (e.g., gastric adenocarcinoma), and mucosa associated lymphoid tissue (MALT) lymphoma.

c. Determining the Level of Expression of β-Defensin 2 (BD2)

The level of expression of BD2 can be measured according to methods well known in the art, and described herein. Levels of expression can be measured at the transcriptional and/or translational levels.

i. mRNA Expression

BD2 expression levels can be detected at the transcriptional level using any method known in the art. At the transcriptional level, mRNA can be detected by, for example, amplification, e.g., PCR, LCR, or hybridization assays, e.g., northern hybridization, RNAse protection, or dot blotting, of mRNA in tissue lysate or in situ in tissue sections, all methods known in the art. The level of mRNA is detected, for example, using directly or indirectly labeled detection agents, e.g., fluorescently or radioactively labeled nucleic acids. These assays are well-known to those of skill in the art and described in, e.g., Ausubel, et al., eds., Current Protocols In Molecular Biology (1987-2010); PRINS and In Situ PCR Protocols (Methods in Molecular Biology), Pellestor, eds, 2006, Human Press; Bagasra and Hansen, In-Situ PCR Techniques, 1997, Wiley-Liss; PCR 3: PCR In Situ Hybridization: A Practical Approach (Vol 3), Herrington and O'Leary, eds., 1998, Oxford University Press; Nuovo, PCR In Situ Hybridization: Protocols and Applications, 1997, Lippincott Williams & Wilkins; In Situ Hybridization Protocols (Methods in Molecular Biology), Darby and Hewitson, eds., 2005, Human Press; Fluorescence In Situ Hybridization (FISH)—Application Guide, Liehr, ed., 2009, Springer Berlin Heidelberg, Schwarzacher and Heslop-Harrison, Practical in Situ Hybridization, 2000, BIOS Scientific Publishers.

Polynucleotides that specifically bind to an expressed BD2 nucleic acid sequence can be labeled with any directly or indirectly detectable moiety, including a fluorophore (i.e., fluoroscein, phycoerythrin, quantum dot, Luminex bead, fluorescent bead), an enzyme (i.e., peroxidase, alkaline phosphatase), a radioisotope (i.e., ³H, ³²P, ¹²⁵I) or a chemiluminescent moiety. Labeling signals can be amplified using a complex of biotin and a biotin binding moiety (i.e., avidin, streptavidin, neutravidin).

The presence or increased presence of BD2 mRNA is indicated by a detectable signal (i.e., a blot, fluorescence, chemiluminescence, color, radioactivity) in an BD2 nucleic acid amplification assay, where the biological sample from the patient (e.g., tissue lysate or tissue section) is contacted with a labeled polynucleotide that specifically hybridizes to a BD2 nucleic acid sequence. This detectable signal can be compared to the signal from a normal or non-cancerous control sample or to a threshold value. In some embodiments, increased expression levels of BD2 mRNA are detected, and the presence or increased susceptibility to a disease mediated by, associated with or secondary to H. pylori infection is indicated, e.g., when the detectable signal of BD2 mRNA expression levels in the test sample is at least about 10%, 20%, 30%, 50%, 75% greater in comparison to the signal of BD2 mRNA in the normal or non-infected control sample or the predetermined threshold value. In some embodiments, an increased expression level of BD2 mRNA is detected, and the presence or an increased susceptibility to a disease mediated by, associated with or secondary to H. pylori infection is indicated, when the detectable signal of BD2 mRNA in the test sample is at least about 1-fold, 2-fold, 3-fold, 4-fold or more, greater in comparison to the signal of BD2 mRNA in the normal or non-infected control sample or the predetermined threshold value. Usually, the sample and control or predetermined threshold levels are from the same tissue types.

In some embodiments, the BD2 mRNA levels are compared with a BD2 mRNA expression level control from tissue known to be infected. In this case, BD2 mRNA expression levels in the test biological sample equivalent to or greater than the positive control sample, known to be infected, are indicative of susceptibility to a disease mediated by, associated with or secondary to H. pylori infection. Usually, the sample and control or predetermined threshold levels are from the same tissue types.

Alternatively, if the BD2 mRNA expression levels in the test biological sample are less than the BD2 mRNA expression levels in the positive cancerous tissue control or the predetermined threshold level, then a diagnosis of susceptibility to a disease mediated by, associated with or secondary to H. pylori infection is generally not indicated. Likewise, if the BD2 mRNA expression levels in the test biological sample are equivalent to or less than a normal or non-cancerous control or the predetermined threshold level, then a diagnosis of susceptibility to a disease mediated by, associated with or secondary to H. pylori infection is not indicated.

In some embodiments, the results of the BD2 mRNA expression level determinations are recorded in a tangible medium. For example, the results of the present diagnostic assays (e.g., the observation of the presence or increased presence of BD2 mRNA) and the diagnosis of whether or not the presence or an increased risk of a disease mediated by, associated with or secondary to H. pylori infection is determined can be recorded, e.g., on paper or on electronic media (e.g., audio tape, a computer disk, a CD, a flash drive, etc.).

In some embodiments, the methods further comprise the step of providing the diagnosis to the patient of whether or not there is the presence or an increased risk of a disease mediated by, associated with or secondary to H. pylori infection in the patient based on the results of the BD2 mRNA expression level determinations.

ii. Protein Expression

BD2 protein expression can be measured using any method known in the art. The level of protein can be detected, for example, using directly or indirectly labeled detection agents, e.g., fluorescently, radioactively or enzymatically labeled antibodies. Expression of BD2 protein can be measured using immunoassays including immunohistochemical staining, Western blotting, ELISA and the like with an antibody that selectively binds to BD2 or a fragment thereof. Detection of the protein using protein-specific antibodies in immunoassays is known in the art (see, e.g., Harlow & Lane, Using Antibodies: A Laboratory Manual (1998); Coligan, et al., eds., Current Protocols in Immunology (1991-2010); Goding, Monoclonal Antibodies: Principles and Practice (3rd ed. 1996); and Kohler & Milstein, Nature 256:495-497 (1975).

Expression levels of BD2 protein in a tissue subject to H. pylori infection and damage therefrom can be detected using any method known in the art. Exemplary methods include tissue lysate detection, Western immunoblot and immunohistochemistry, as demonstrated herein. The tissue sample can be from the tissue affected by the H. pylori infection. In various embodiments, the solid tissue sample is gastric tissue or intestinal tissue. Alternatively, the biological tissue sample can be a fluid tissue sample, e.g., from tears, urine, blood or saliva, in instances where the BD2 protein levels correlate to the BD2 gene copy number.

For detection of the expression levels of BD2 proteins in a tissue sample, a tissue sample is incubated with an antibody that specifically binds to BD2 protein under conditions (i.e., time, temperature, concentration of sample) sufficient to allow specific binding. If appropriate or desired, the tissues can be fixed (e.g., in formaldehyde) and permeabilized prior to incubation with antibody, to allow the antibody to access the BD2 protein. The anti-BD2 antibodies can be exposed to a tissue sample for about 0.5, 1.0, 1.5, 2.0, 2.5, 3.0 hours, or overnight, about 8, 10 or 12 hours, as appropriate. However, incubation time can be more or less depending on, e.g., the composition of the antigen, the dilution of the sample and the temperature for incubation. Incubations using less diluted samples and higher temperatures can be carried out for shorter periods of time. Incubations are usually carried out at room temperature (about 25° C.) or at biological temperature (about 37° C.), and can be carried out in a refrigerator (about 4° C.). Washing to remove unbound sample before addition of a secondary antibody is carried according to known immunoassay methods.

Labeled secondary antibodies are generally used to detect antibodies or autoantibodies in a sample that have bound to one or more LDH polypeptides, antigenic fragments thereof or LDH mimeotopes. Secondary antibodies bind to the constant or “C” regions of different classes or isotypes of immunoglobulins—IgM, IgD, IgG, IgA, and IgE. Usually, a secondary antibody against an IgG constant region is used in the present methods. Secondary antibodies against the IgG subclasses, for example, IgG1, IgG2, IgG3, and IgG4, also find use in the present methods. Secondary antibodies can be labeled with any directly or indirectly detectable moiety, including a fluorophore (i.e., fluoroscein, phycoerythrin, quantum dot, Luminex bead, fluorescent bead), an enzyme (i.e., peroxidase, alkaline phosphatase), a radioisotope (i.e., ³H, ³²P ,¹²⁵I) or a chemiluminescent moiety. Labeling signals can be amplified using a complex of biotin and a biotin binding moiety (i.e., avidin, streptavidin, neutravidin). Fluorescently labeled anti-human IgG antibodies are commercially available from Molecular Probes, Eugene, Oreg. Enzyme-labeled anti-human IgG antibodies are commercially available from Sigma-Aldrich, St. Louis, Mo. and Chemicon, Temecula, Calif.

The method of detection of the levels of BD2 protein in a sample will correspond with the choice of label of the secondary antibody. For example, if tissue lysates containing BD2 protein are transferred onto a membrane substrate suitable for immunoblotting, the detectable signals (i.e., blots) can be quantified using a digital imager if enzymatic labeling is used or an x-ray film developer if radioisotope labeling is used. Likewise, tissue samples subject to immunohistochemistry can be evaluated using immunofluorescence microscopy or a scanning microscope and automated scanning software capable of detecting and quantifying fluorescent, chemiluminescent, and/or colorimetric signals. Such methods of detection are well known in the art and are described herein.

General immunoassay and immunohistochemical techniques are well known in the art. Guidance for optimization of parameters can be found in, for example, Wu, Quantitative Immunoassay: A Practical Guide for Assay Establishment, Troubleshooting, and Clinical Application, 2000, AACC Press; Principles and Practice of Immunoassay, Price and Newman, eds., 1997, Groves Dictionaries, Inc.; The Immunoassay Handbook, Wild, ed., 2005, Elsevier Science Ltd.; Ghindilis, Pavlov and Atanassov, Immunoassay Methods and Protocols, 2003, Humana Press; Harlow and Lane, Using Antibodies: A Laboratory Manual, 1998, Cold Spring Harbor Laboratory Press; Immunoassay Automation: An Updated Guide to Systems, Chan, ed., 1996, Academic Press; Dabbs, Diagnostic Immunohistochemistry: Theranostic and Genomic Applications, 2010, Saunders; Renshaw, Immunohistochemistry: Methods Express Series, 2007, Scion Publishing Ltd.; and Buchwalow and Böcker, Immunohistochemistry: Basics and Methods, 2010, Springer.

The presence or increased presence of BD2 protein is indicated by a detectable signal (i.e., a blot, fluorescence, chemiluminescence, color, radioactivity) in an immunoassay or immunohistochemical assay, where the biological sample from the patient is contacted with antibody or antibody fragment that specifically binds to BD2 protein. This detectable signal can be compared to the signal from a normal or non-infected control sample or to a threshold value. In some embodiments, increased expression levels of BD2 protein are detected, and the presence or increased risk of a disease mediated by, associated with or secondary to H. pylori infection is indicated, e.g., when the detectable signal of BD2 protein expression levels in the test sample is at least about 10%, 20%, 30%, 50%, 75% greater in comparison to the signal of BD2 protein in the normal or non-infected control sample or the predetermined threshold value. In some embodiments, increased expression levels of BD2 protein is detected, and the presence or an increased risk of a disease mediated by, associated with or secondary to H. pylori infection is indicated, when the detectable signal of BD2 protein in the test sample is at least about 1-fold, 2-fold, 3-fold, 4-fold or more, greater in comparison to the signal of BD2 protein in the normal or non-infected control sample or the predetermined threshold value. Usually, the sample and control or predetermined threshold levels are from the same tissue types.

In some embodiments, the BD2 protein levels are compared with a BD2 protein expression level control from tissue known to be infected or susceptible to a disease mediated by, associated with or secondary to H. pylori infection. In this case, BD2 protein expression levels in the test biological sample equivalent to or greater than the positive control sample, known to be infected or susceptible to a disease mediated by, associated with or secondary to H. pylori infection, are indicative of susceptibility to a disease mediated by, associated with or secondary to H. pylori infection. Usually, the sample and control or predetermined threshold levels are from the same tissue types.

Alternatively, if the BD2 protein expression levels in the test biological sample are less than the BD2 protein expression levels in the positively infected tissue control or the predetermined threshold level, then a diagnosis of susceptibility to a disease mediated by, associated with or secondary to H. pylori infection is generally not indicated. Likewise, if the BD2 protein expression levels in the test biological sample are equivalent to or less than a normal or non-infected control or the predetermined threshold level, then a diagnosis of susceptibility to a disease mediated by, associated with or secondary to H. pylori infection is not indicated.

In some embodiments, the results of the BD2 protein expression level determinations are recorded in a tangible medium. For example, the results of the present diagnostic assays (e.g., the observation of the presence or increased presence of BD2 protein) and the diagnosis of whether or not the presence or an increased risk of a disease mediated by, associated with or secondary to H. pylori infection is determined can be recorded, e.g., on paper or on electronic media (e.g., audio tape, a computer disk, a CD, a flash drive, etc.).

In some embodiments, the methods further comprise the step of providing the diagnosis to the patient of whether or not there is the presence or an increased susceptibility to a disease mediated by, associated with or secondary to H. pylori infection in the patient based on the results of the BD2 protein expression level determinations.

d. Delivering Appropriate Diagnosis and/or Therapy to Subject

In various embodiments, the methods further comprise the step of providing to the patient a diagnosis of increased susceptibility to a disease mediated by, associated with or secondary to H. pylori infection, or lack thereof, depending on the results of the determinations of BD2 gene copy number and/or levels of BD2 expression.

Upon a positive diagnosis of increased susceptibility to a disease mediated by, associated with or secondary to H. pylori infection or confirmation of a diagnosis of susceptibility to a disease mediated by, associated with or secondary to H. pylori infection, the present methods may further include the step of determining an appropriate therapeutic regimen for the patient, and/or administering an appropriate therapy based on the diagnosis of susceptibility to a disease mediated by, associated with or secondary to H. pylori infection.

The appropriate therapeutic regimen can be based on present clinical treatments available to patients diagnosed H. pylori infection, e.g., administration of an antibiotic.

In some embodiments, the established therapy regimen against H. pylori infection is co-administered with an inhibitory nucleic acid that inhibits expression of BD2, as described herein.

4. Kits

The present invention also includes an BD2-detection reagent, e.g., a nucleic acid that specifically binds to or identifies one or more BD2 nucleic acids, including oligonucleotide sequences which are complementary to a portion of a BD2 nucleic acid, or an antibody that binds to one or more proteins encoded by an BD2 nucleic acid. The detection reagents can be packaged together in the form of a kit. For example, the detection reagents can be packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, USB drive, etc.) for carrying out the assay and correlating increased gene copy number of the BD2 gene and/or increased expression of BD2 mRNA with an affirmative diagnosis of increased susceptibility to a disease mediated by, associated with or secondary to an H. pylori infection can also be included in the kit. The assay format of the kit can be, e.g., a microarray analysis, PCR, Northern hybridization or a sandwich ELISA, both of which are known in the art. See, for example, Sambrook and Russell, Molecular Cloning: A Laboratory Manual, 3^{rd} Edition, 2001, Cold Spring Harbor Laboratory Press; and Harlow and Lane, Using Antibodies, supra.

For example, a BD2 detection reagent can be immobilized on a solid matrix, for example, a porous strip, to form at least one BD2 detection site. The measurement or detection region of the porous strip can include a plurality of sites, each containing a nucleic acid. A test strip can also contain sites for negative and/or positive controls. Alternatively, control sites can be located on a separate strip from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of BD2 present in the sample. The detection sites can be configured in any suitably detectable shape and are typically in the shape of a bar or dot spanning the width of a test strip.

In various embodiments, the kits contain reagents that allow for simultaneously detecting, in a biological specimen containing genomic DNA, the ratio of gene copy numbers for a reference gene (present in an invariant number of copies per genome, such as 2 copies per diploid genome, or perhaps 4 copies per diploid genome) and the BD2 gene (present in an unknown GCN). The kit can contain oligonucleotide primers for use in the detection of GCN of the BD2 gene and at least one reference gene. The kit can further contain instructions for determining GCN, e.g., by multiplex PCR. In some embodiments, the primers provided in the kit are labeled so that the PCR product from each gene can be distinguishable by, e.g., a colorimetric or fluorescent label, so that the ratio of products could be detectable by a change in color.

For example, the kits can contain reagents for performing gene-specific PCR assays, utilizing a first primer pair that hybridizes to the BD2 gene labeled with a first fluorescent label and a second primer pair that hybridizes to a reference gene labeled with a second fluorescent label. Simultaneous (multiplex) PCR produces two reaction products, each with a different fluorescent tag. The ratio of two products is detected, e.g., by a cumulative fluorescence signal, and yields a useful estimate of BD2 GCN. To provide a non-limiting illustration, if the reference gene product was labeled red and the BD2 product was labeled green, a multiplex PCR reaction that had a yellow product would indicate that the BD2 GCN equals reference GCN. A more red product would indicate lower BD2 GCN and a more green product indicates higher BD2. Exemplary reference genes that find use include so-called housekeeping genes and genes that do not have pseudogenes, e.g., TATA box binding protein (“TBP”), myeloperoxidase, β-globin. A reference gene with 4 copies per diploid genome is also useful, because the yellow to green transition would occur at the point of increased risk for BD2 GCN. Exemplary genes with 4 copies per diploid genome include without limitation, e.g., CYP2D6, complement component C4 (C4), and deleted in azoospermia 1 (DAZ1).

Because the PCR is done in a single tube including the BD2 test and gene reference controls, the actual number of GCN can be estimated based on the cumulative fluorescent signal. The relative value of BD2 GCN in comparison to the reference gene GCN, based on the fluorescent product, is sufficient to allow a practitioner to make and render a diagnosis or prognosis of risk of developing a disease associated with or mediated by H. pylori infection. To expand upon the example provided above, a “greenish” fluorescent product would be sufficient to warrant antibiotic treatment to eradicate H. pylori in the “at risk” patient. In other words, by simultaneously evaluating the GCN of BD2 with a reference gene of known copy number, information that the BD2 GCN is “high” is sufficient without the requirement of obtaining a specific GCN value.

In other embodiments, the invention provides kits containing an inhibitory nucleic acid that specifically hybridizes to and inhibits expression of a BD2 nucleic acid and instructions for administration to a patient to reduce, inhibit or prevent a disease mediated by, associated with or secondary to H. pylori infection, e.g., peptic ulcer, non-ulcer dyspepsia, gastric cancer (e.g., gastric adenocarcinoma), and/or mucosa associated lymphoid tissue (MALT) lymphoma.

5. Screening for Agents That Inhibit BD2 Expression

The invention further provides for methods of identifying agents that inhibit BD2 expression levels or activity. An agent that inhibits the expression of BD2 genes or BD2 proteins can be identified by contacting a test cell population expressing a BD2 protein with a test agent and then determining the expression level of BD2, at the transcriptional or translational level. A decrease in the level of expression of BD2 mRNA or protein in the presence of the agent as compared to the expression or activity level in the absence of the test agent indicates that the agent is an inhibitor of BD2 overexpression and therefore useful in inhibiting diseases associated with, correlative with or caused by the overexpression of BD2.

The test cell population can be comprised of any cells expressing or overexpressing a BD2 encoding nucleic acid. For example, the test cell population can contain epithelial cells, for example, cells gastric or intestinal tissue. Furthermore, the test cell population can be an immortalized cell line. Alternatively, the test cell population can be cells which have been transfected with a BD2 nucleic acid or which have been transfected with a regulatory sequence (e.g. promoter sequence) operably linked to a reporter gene.

The agent that inhibits BD2 expression can be, for example, an inhibitory oligonucleotide (e.g., an antisense oligonucleotide, an siRNA, a micro RNA, a ribozyme), an antibody, a polypeptide, a small organic molecule. Screening for agents can be carried out using high throughput methods, by simultaneously screening a plurality of agents using multiwell plates (e.g., 96-well, 192-well, 384-well, 768-well, 1536-well). Automated systems for high throughput screening are commercially available from, for example, Caliper Life Sciences, Hopkinton, Mass. Small organic molecule libraries available for screening can be purchased, for example, from Reaction Biology Corp., Malvern, Pa.; TimTec, Newark, Del.

The level of BD2 expression in a test sample can be compared to the BD2 expression from a control sample (e.g., a cell that has not been contacted with the test agent) or to a threshold value. Test agents of interest decrease, reduce or inhibit BD2 expression levels in the test sample by at least about 10%, 20%, 30%, 50%, 75%, or greater, in comparison to the BD2 expression in a control sample or the predetermined threshold value. In some embodiments, a decreased BD2 expression is decreased, reduced or inhibited by at least about 1-fold, 2-fold, 3-fold, 4-fold or more, in comparison to the BD2 expression in the control sample or the predetermined threshold value. Usually, the sample and control or predetermined threshold levels are from the same tissue types.

6. Methods of Inhibiting BD2 Expression

The invention further provides methods of reducing, inhibiting or preventing the growth of a cancer cell or a tumor the overexpresses BD2 by contacting the cancer cell or tumor that overexpresses BD2 with an inhibitory nucleic acid (e.g., antisense RNA, ribozyme, short inhibitory RNA, micro RNA, etc.) that selectively hybridizes to a BD2 nucleic acid sequence. The cancer cell or tumor can be contacted in vitro or in vivo.

Relatedly, the invention further provides reducing or inhibiting the expression of a BD2 nucleic acid in a gastric tissue or intestinal tissue cell by contacting the cell with a BD2 inhibitory nucleic acid. The gastric tissue or intestinal tissue cell may be in vitro or in vivo.

A nucleic acid molecule complementary to at least a portion of the BD2 gene can be used to inhibit BD2 gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of BD2, siRNAs to the gene encoding BD2 can be specifically designed using computer programs. A program, siDESIGN from Dharmacon, Inc. (Lafayette, Co.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 by siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplary vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO:9). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence.

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the inhibitory nucleic acid sequence is substantially complementary to the target sequence (e.g., at least about 95%, 96%, 97%, 98% or 99% complementary). In certain embodiments, the inhibitory nucleic acid sequence is exactly complementary to the target sequence. The inhibitory nucleic acid polynucleotides may also include, however, nucleotide substitutions, additions, deletions, transitions, transpositions, or modifications, or other nucleic acid sequences or non-nucleic acid moieties so long as specific binding to the relevant target sequence corresponding to the BD2 gene is retained as a functional property of the polynucleotide. In one embodiment, the inhibitory nucleic acid molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591).

Antisense molecules can be designed by methods known in the art. For example, Integrated DNA Technologies (Coralville, Iowa) makes available a program found on the worldwide web “biotools.idtdna.com/antisense/AntiSense.aspx”, which will provide appropriate antisense sequences for nucleic acid sequences up to 10,000 nucleotides in length.

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a BD2 gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found on the worldwide web at “nature.com/news/2005/050418/full/050418-6.html. ”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4):1290-7 (2005); Robins and Padgett, Proc Natl Acad Sci USA. 102(11):4006-9 (2005); Brennecke et al., PLoS Biol. 3(3):c85 (2005). Methods of designing miRNAs are known. See, e.g., Zeng et al., Methods Enzymol. 392:371-80 (2005); Krol et al., J Biol Chem. 279(40):42230-9 (2004); Ying and Lin, Biochem Biophys Res Commun. 326(3):515-20 (2005).

The inhibitory nucleic acid can be targeted to the tissue to be treated. Targeting can be accomplished using any method known in the art. Targeting of inhibitor nucleic acids is discussed, e.g., in Jackson and Linsley, Nat Rev Drug Discov. (2010) 9(1):57-67; Pappas, et al., Expert Opin Ther Targets. 2008 12(1):115-27; Li, et al., AAPS J. (2009) 11(4):747-57.

### EXAMPLES

The following examples are offered to illustrate, but not to limit the claimed invention.

### Example 1

The non-human primate model recapitulates essential features of human infection with H. pylori. Like humans living in developing countries, socially housed macaques are commonly infected with H. pylori early in life (Solnick, et al., J. Clin. Microbiol. (2003) 41:5511-5516). Prevalence is 40% by 12 wks of age and is ubiquitous at one year. Although natural infection among socially housed monkeys supports the relevance of the model (no other animals are naturally infected), it is an impediment to experimental infection. Specific pathogen (H. pylori)-free (SPF) macaques were developed by hand rearing them in the nursery beginning the day of birth, and then experimentally infecting them with strain J166, a cag PAI positive strain isolated from a patient with peptic ulcer disease that is adapted to colonization of rhesus monkeys (Dubois, et al., Infect. Immun. (1996) 64:2885-2891). Experimental infection with this wild type H. pylori strain induces a histologic gastritis that closely mimics that seen in humans. The rhesus macaque model has been used to address a variety of questions regarding the pathogenesis of H. pylori (Boonjakuakul, et al, Infect. Immun. (2005) 73:4895-4904; Hornsby, et al., Gastroenterol (2008) 134:1049-1057; Solnick, et al., J. Clin. Microbiol. (2003) 41:5511-5516; Solnick, et al., Proc. Natl. Acad. Sci. U.S.A. (2004) 101:2106-2111). Although clinical endpoints of infection in humans, such as peptic ulcer, gastric adenocarcinoma, and MALT lymphoma, are sometimes seen in macaques, their connection with H. pylori has not been systematically studied.

### Example 2

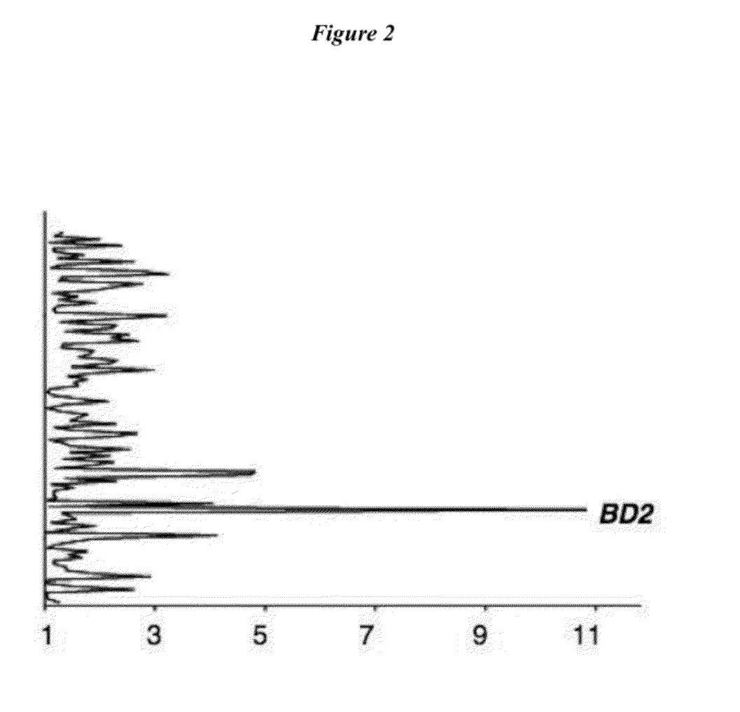

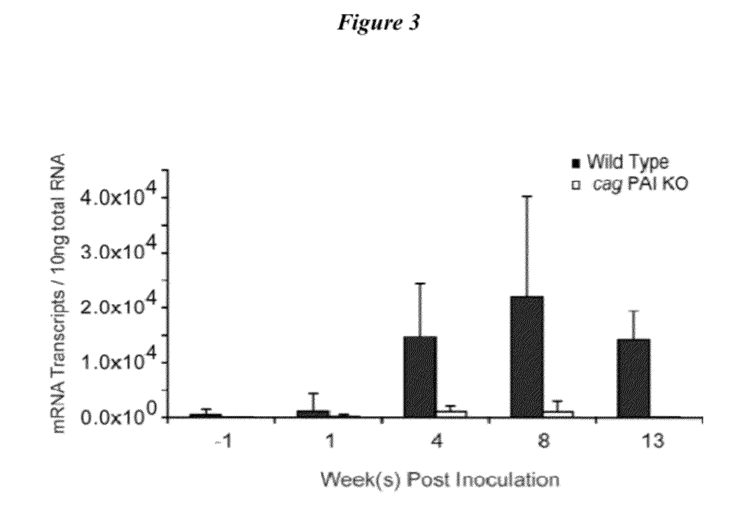

Colonization of rhesus macaques with H. pylori induces expression of BD2. SPF rhesus macaques were inoculated with wild-type (WT) H. pylori and with an isogenic knockout (KO) strain in which the cag PAI was deleted (Hornsby, et al., Gastroenterol (2008) 134:1049-1057). Quantitative cultures of three antral biopsies at 1, 4, 8, and 13 wks post inoculation showed that all challenged animals (but not controls) were infected, typically with 10⁵ to 10⁷ CFU/g of gastric tissue. Sections of gastric histopathology demonstrated that monkeys infected with WT H. pylori had gastritis typical of that seen in humans, while the inflammation was much reduced in the KO infected animals. Microarray analysis was performed on gastric mucosa at 13 weeks post inoculation and on uninfected control animals (FIG. 2). A key observation was the induction of BD2, which was induced ˜11-fold. This effect was dependent upon the cag PAI, as minimal change in BD2 expression was detected in H. pylori PAI KO-infected animals at any time point. Although counterintuitive, these results suggest that one function of the cag PAI during H pylori infection is to mediate induction of BD2, an innate immune antimicrobial effector molecule. These data were confirmed by real time quantitative RT-PCR (FIG. 3) and by immunohistochemistry (Hornsby, et al., Gastroenterol (2008) 134:1049-1057). One-way ANOVA and pairwise t tests demonstrated that BD2 gene expression was significantly greater in the WT-infected animals compared to cag PAI KO-challenged animals and uninoculated controls. There were no differences in expression of β-actin among the three groups, which served as a quantitative control for the quality of RNA.

### Example 3

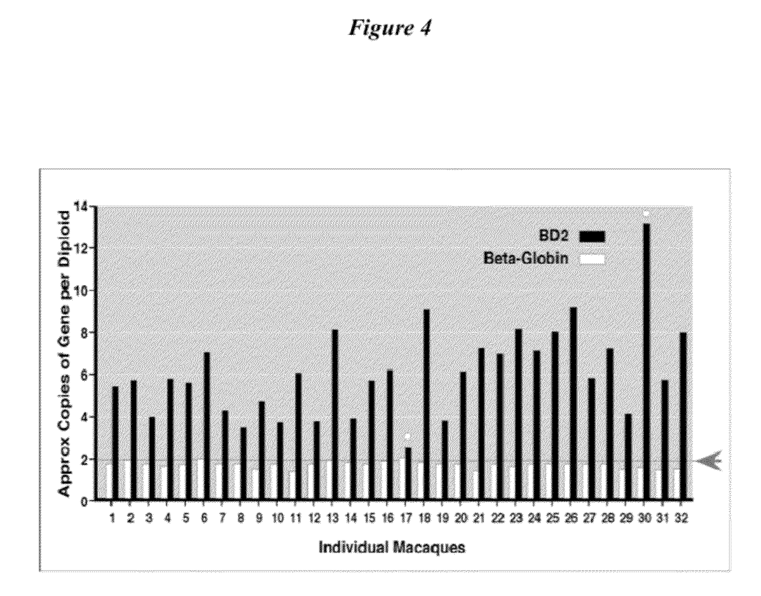

GCN variation in BD2. Both comparative genomic analysis and locus-specific analysis of primate β-defensins genes suggests that, unlike in rodents, there has not been rapid duplication and divergence of β-defensin genes in primates. Instead, GCN seems to have been maintained across primates and concerted evolution occurred between paralogous copies of β-defensin genes (Hornsby, et al., Gastroenterol (2008) 134:1049-1057). Rhesus macaques were selected as a non-human primate to determine the role of BD2 GCN in the host response to H. pylori infection. BD2 GCN variation was detected in 32 randomly selected rhesus macaques. Genomic DNA was extracted from blood samples, and quantitative real-time PCR was used to determine BD2 GCN (FIG. 4). One set of primers was used to detect all copies. The following exemplary human primers were used:

(SEQ ID NO: 1)
BD2 Forward: AGGCGATACTGACACAGGGTTTGT
(SEQ ID NO: 2)
BD2 Reverse: GGAGACCACAGGTGCCAATTTGTT
(SEQ ID NO: 3)
BD2 Forward: ATCAGCCATGAGGGTCTTGT
(SEQ ID NO: 4)
BD2 Reverse: GGAGGGGAATGAGAGGAGAC

The amount of specific product reflects the GCN when the reactions are controlled to be quantitative, e.g., by determining the amount of BD2 gene amplification in comparison to the amount of amplification from a reference gene. In the present assays, a reference gene present in 2 copies per diploid genome was used, although a reference gene present in greater than 2 copies per diploid genome, e.g., 4 or 6 copies per diploid genome would also be useful. If the amount of product amplified using the BD2 specific primers is equivalent to the amount of product amplified using primers specific for the reference gene known to have 2 copies per diploid genome, it is determined that BD2 in that individual is present in 2 copies per diploid (GCN=2 for references and BD2). Similarly, if the amount of product amplified using the BD2 specific primers is greater than the amount of product amplified using primers specific for the reference gene known to have 2 copies per diploid genome, the GCN of BD2 can be determined or estimated by determining the ratio of the amount of product amplified using the BD2-specific primers in comparison to the amount of product amplified using the primers specific for the reference gene. In qPCR the reaction is monitored at every cycle. The cycle at which the product is first at the threshold of detection is the “crossover point” and can be used to estimate the amount of target sequence in the original DNA added to the tube. In the present assays, the same amount of genomic DNA was added to the tubes. Samples with high GCN will cross the threshold sooner (at an earlier cycle) than those samples with lower GCN. Put another way, the earlier crossover point reflects a higher number of target sequences (gene number) per a defined amount of genomic DNA (for example, 10 ng).

As a control and reference, values for β-globin were used as a diploid gene. In contrast to the GCN of ˜2 per diploid consistently observed for β-globin, the BD2 GCN varied from ˜2 to ˜13 among these animals. Other reference genes commonly used include myeloperoxidase ((MPO), Ensembl:ENSG00000005381) and TATA box-binding protein ((TBP), Ensembl:ENSG00000112592). This range of BD2 GCN is similar to that observed in humans (2-12 copies/diploid). Interestingly, PCR and 3′-rapid amplification of cDNA ends revealed that, in contrast to a single isoform of human BD2, a group of highly similar BD2 orthologs is present in rhesus macaques (Hornsby, et al., Gastroenterol (2008) 134:1049-1057). The qRT-PCR assay using primers designed to detect all of these BD2-like transcripts demonstrated that this group of β-defensins was induced to high levels in a cag PAI-dependent manner (FIG. 3).

The deduced peptide sequences (designated BD2-like 1-5) were about 75% identical to human BD2 (Hornsby, et al., Gastroenterol (2008) 134:1049-1057). BD2-like 1, BD2-like 2, BD2-like 3, BD2-like 3, BD2-like 4 and BD2-like 5 are nearly identical sequences of the same gene (officially termed “paralogs”). This investigation of BD2 GCN variation (FIG. 4) provided a clue of the origin of some of the sequence polymorphism. To determine whether individual macaques harbored extensive single nucleotide variants of BD2, BD2 mRNA was cloned from two individual macaques, one with 2 copies of BD2/diploid and one with 13 copies of BD2/diploid genome (FIG. 4, #'s 17 and 30). Two variants (BD2-L7 and -L8) were found in macaque #17, which harbored two BD2 gene copies per diploid. Two other variants were detected at the deduced protein level, BD2-L1 and -L6 in macaque #30, which harbored 13 copies of BD2. Within the limited number of clones identified, five paralogs were accounted for when non-coding variations were taken into account. These data highlight that genetic variation in BD2 is present in rhesus macaques, in terms of both GCN variation and single nucleotide variation. They also suggest that the multiple variants are indeed expressed in tissue. This contrasts somewhat from humans, where BD2 GCN is observed, but little or no single nucleotide variation is evident.

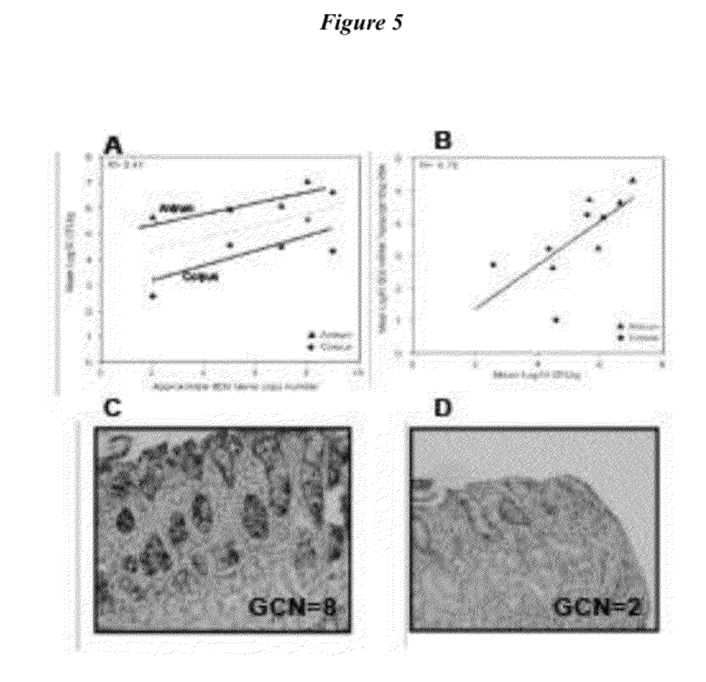

High BD2 GCN is associated with increased H. pylori bacterial load. The H. pylori challenge of rhesus macaques (N=5) was repeated and the relationship between BD2 GCN and H. pylori bacterial load in the gastric antrum and corpus was examined. H. pylori colony forming units (CFU) was positively correlated (R=0.47) with BD2 GCN (FIG. 5A). A positive correlation (R=0.70) between H. pylori CFU and BD2 mRNA transcript abundance was also found (FIG. 5B), which was confirmed by immunohistochemistry with anti-BD2 antibody staining of gastric tissue from an animal with 8 BD2 gene copies (FIG. 5C) compared to one with 2 BD2 gene copies (FIG. 5D). These data are clearly consistent with the conclusion that BD2 increases the fitness of H. pylori, perhaps by selectively suppressing competing gastric biota.

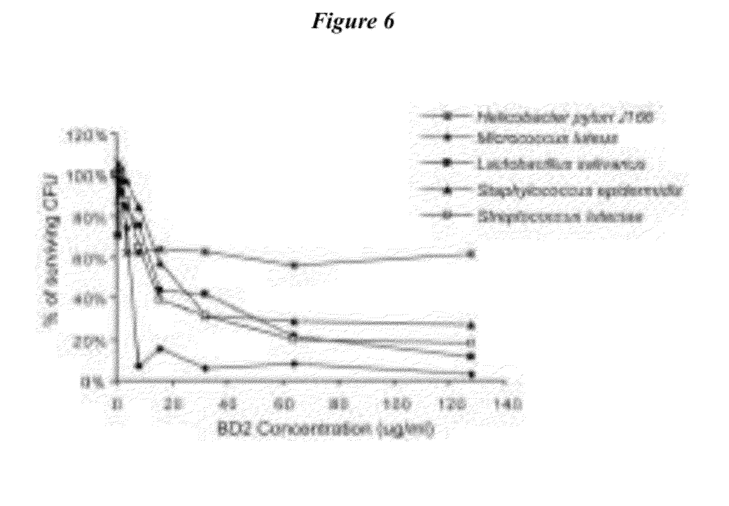

Antimicrobial activity of recombinant rhesus BD2. The antimicrobial activity of recombinant rhesus BD2 against H. pylori was determined and other bacteria that are isolated routinely from gastric biopsies of rhesus macaques were selected, including Micrococcus, Lactococcus, Streptococcus, and Staphylococcus. Bacteria were grown to mid-logarithmic phase in the appropriate liquid culture medium, incubated in serial dilutions of BD2, and quantified by serial plate counts. The results (FIG. 6), while preliminary, suggest that H. pylori is less sensitive to BD2 than other members of the gastric microbial community.

Summary. Experimental infection of primates with H. pylori induces a PAI-dependent gastritis and induction of mucosal BD2 that mimics that seen in humans. Like in humans, rhesus BD2 GCN is highly variable and is positively correlated with H. pylori bacterial load. This observation, together with the finding that BD2 has relatively little activity against H. pylori, suggests that BD2 increases the fitness of H. pylori and increase the risk of H. pylori-associated clinical disease.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

What is claimed is:

1. A method of determining an increased susceptibility of an individual to a disease condition mediated by an Helicobacter pylori infection comprising determining in a biological sample from the individual the gene copy number (GCN) of the β-defensin 2 (BD2) gene, wherein a high GCN of the BD2 gene is indicative of an increased susceptibility of the individual to the disease condition mediated by the H. pylori infection.

2. The method of claim 1, wherein a GCN of the BD2 gene that is 5 or higher is indicative of an increased susceptibility of the individual to the disease condition mediated by the H. pylori infection.

3. The method of claim 1, wherein the disease condition mediated by the H. pylori infection is selected from the group consisting of peptic ulcer, non-ulcer dyspepsia, gastric cancer, and mucosa associated lymphoid tissue (MALT) lymphoma.

4. The method of claim 1, wherein the GCN is determined by a method selected from the group consisting of PCR, fluorescent in situ hybridization, multiplex amplifiable probe hybridization (MAPH), multiplex ligation-dependent probe amplification (MLPA), dynamic allele-specific hybridization (DASH), array hybridization and mass spectroscopy.

5. The method of claim 1, further comprising the step of determining the expression of BD2 in gastric tissue in the individual, wherein the increased expression of BD2 in gastric tissue is indicative of an increased susceptibility of the individual to the disease condition mediated by the H. pylori infection.

6. The method of claim 5, wherein the expression level of the BD2 gene is determined.

7. The method of claim 5, wherein the expression level of the BD2 protein is determined.

8. The method of claim 1, further comprising the step of determining bacterial load of H. pylori in the individual, wherein a high bacterial load is indicative of an increased susceptibility of the individual to the disease condition mediated by the H. pylori infection.

9. A method of determining an increased susceptibility of an individual to a disease condition mediated by an Helicobacter pylori infection comprising determining in a biological sample from the individual the expression level of BD2, wherein a high expression level of BD2 is indicative of an increased susceptibility of the individual to the disease condition mediated by the H. pylori infection.

10. The method of claim 9, wherein the expression level of the BD2 gene is determined.

11. The method of claim 9, wherein the expression level of the BD2 protein is determined.

12. The method of claim 9, wherein the biological sample is gastric tissue.

13. The method of claim 9, wherein the disease condition mediated by the H. pylori infection is selected from the group consisting of peptic ulcer, non-ulcer dyspepsia, gastric cancer, and mucosa associated lymphoid tissue (MALT) lymphoma.

14. The method of claim 9, further comprising determining in a biological sample from the individual the gene copy number (GCN) of the β-defensin 2 (BD2) gene, wherein a high GCN of the BD2 gene is indicative of an increased susceptibility of the individual to the disease condition mediated by the H. pylori infection.

15. The method of claim 14, wherein a GCN of the BD2 gene that is 5 or higher is indicative of an increased susceptibility of the individual to the disease condition mediated by the H. pylori infection.

16. The method of claim 9, further comprising the step of determining bacterial load of H. pylori in the individual, wherein a high bacterial load is indicative of an increased susceptibility of the individual to the disease condition mediated by the H. pylori infection.

17. A method for identifying a candidate for treatment of a disease condition mediated by an Helicobacter pylori infection, comprising: a) providing a biological sample of from a candidate subject; b) detecting a gene copy number (GCN) for the BD2 gene in said biological sample; and c) identifying said candidate subject as suitable for treatment with an inhibitor of BD2.

18. The method of claim 17, wherein the subject has an H. pylori infection.

19. The method of claim 17, wherein the biological sample is blood or saliva.

20. The method of claim 17, wherein a GCN of the BD2 gene that is 5 or higher identifies said candidate subject as suitable for treatment with an inhibitor of BD2.
